(19) United States
(12) Patent Application Publication
(10) Pub. No.: US 2017/0333192 A1
(43) Pub. Date: Nov. 23, 2017

(54) Non-metal Ball-headed Hip Joint Prosthesis

(71) Applicants: Diange Zhou, Tianjin City (CN); Nian Liu, Tianjin City (CN)

(72) Inventors: Diange Zhou, Tianjin City (CN); Nian Liu, Tianjin City (CN)

(21) Appl. No.: 15/405,333

(22) Filed: Jan. 13, 2017

(30) Foreign Application Priority Data
May 17, 2016 (CN) 201610325901.7

(51) Int. Cl. A61F 2/34; A61F 2/36; A61F 2/32; A61F 2/30

(52) U.S. Cl. CPC A61F 2/34; A61F 2/3672; A61F 2/3609; A61F 2002/3619; A61F 2002/3611; A61F 2002/345; A61F 2002/3225; A61F 2002/30654; A61F 2002/3065; A61F 2002/3654; A61F 2310/00179; A61F 2002/3429

(57) ABSTRACT

This invention revolves around medical devices, especially the non-metal ball-headed hip joint prosthesis, including acetabular cup, non-metal femoral head and femoral stem prostheses. In this invention, the acetabular cup is combined with the lining as a metal cup, and the traditional metal or ceramic ball head is replaced by non-metal femoral head prosthesis with metal inner core.

### TECHNICAL FIELD

This invention revolves around medical apparatus, especially the non-metal ball-headed hip joint prosthesis.

### BACKGROUND TECHNOLOGY

As shown by FIG. 1, traditional total jip joint prosthesis is comprised of metal acetabular cup, polyethylene lining, femoral head, and femoral stem. Metal acetabular cup 1 is combined with lining 2 as a whole, and the lining 2 has an inner spherical surface, which matches the external spherical surface of femoral head 3. In this way, femoral head 3 which moves within lining 2 can facilitate the movement of hip joint prosthesis.

Human's normal hip joint is characterized by abduction and anteversion. The articular capsule, muscle and ligament around hip joint can contribute to the stability of hip joint. After the operation of artificial hip joint, the articular capsule, muscle and ligament stabilizing hip joint will not completely return to normal. In view of this, existing lining products are designed to protrude from one outer side of metal acetabular cup so that the femoral head prosthesis will not prolapse from acetabular cup prosthesis to result in dislocation. Meanwhile, most of existing lining products are made of ultra-high molecular weight polyethylene (UHMWPE). Given its strength and abrasion, all factories are required to design the lining whose thickness is no less than 5 mm.

When human body's acetabulum model is confirmed, the thicknesses of metal acetabular cup and lining, as well as the diameter of femoral head, need to suit the acetabulum. Thus, the thicknesses of metal acetabular cup and lining are required to be suitably designed. This will occupy much space of femoral head. Particularly, when small-sized acetabular cup is used, femoral head should be designed based on a smaller diameter (such as 28 mm or even 22 mm). This will lead to the results below: (1) there is a decrease in contact area of the articular surface but an increase in abrasion of prostheses; (2) the small-diameter ball head will be easily dislocated; (3) the motion range of joint prosthesis will be influenced and no greater than 90°. The FIG. 2 and FIG. 3 show the two motion limits of traditional total hip joint prosthesis, which, however, are far from meeting the needs of human body because the motion range of human's physiological hip joint exceeds 120°. If the diameter of femoral head is indeed required to increase, higher bone mass within acetabulum needs to be diminished. However, the increase in acetabulum model will lead to greater bone losses.

### INVENTION CONTENTS

The technical problem to be solved by this invention is to conquer the deficiencies of the small-diameter ball head in existing technologies and products: large decline in bone mass, severe abrasion and easy dislocation. This invention proposes a method which changes the articular surface, i.e., replaces not only the original lining with a simple metal acetabular cup which has a smooth inner surface, but also the original metal femoral head with the non-metal femoral head which has metal inner core. In this way, the metal acetabular cup will decrease its thickness greatly so that more room will be provided for the increase in the diameter of the ball head and lower bone mass will be decreased during the lateral installation of the ball head in acetabulum.

This invention is implemented based on the technical scheme below:

Non-metal ball-headed hip joint prosthesis includes acetabular cup, non-metal femoral head and femoral stem prostheses. The acetabular cup prosthesis has an inner spherical surface, which matches the external spherical surface of non-metal femoral head prosthesis. The non-metal femoral head prosthesis which moves within the acetabular cup prosthesis is equipped with a tapered inner core. A tapered locked connection will take shape when the femoral stem prosthesis is inserted into the tapered inner core of the non-metal femoral head prosthesis.

The femoral stem prosthesis has a rough surface

The rough surface at the outside of the femoral stem prosthesis undergoes the cutting process at its right angle.

The non-metal femoral head prosthesis is made of highly crosslinked polyethylene

The non-metal femoral head prosthesis is made of polyether ether ketone (PEEK).

The tapered inner core is made of metal.

The tapered inner core is made of ceramic.

Below is the beneficial result of this invention.

This invention replaces not only the original lining at the side of acetabulum with a simple metal acetabular cup which has a smooth inner surface, but also the metal femoral head with the non-metal ball head which has metal inner core. The diameter of ball head will increase (particularly, the patients with small-sized acetabulum can still make use of large-diameter ball-headed prosthesis) as the thickness of acetabular cup decreases. In this way, the installation of large-diameter ball head will increase the motion range of hip joint as well as meet the requirements such as (1) the lateral bone bass of acetabulum remains unchanged; (2) the abrasion will be reduced; and (3) the dislocation will be prevented.

### DESCRIPTION ON FIGURES

FIG. 1. Structural Diagram for the Traditional Total Hip Joint Prosthesis

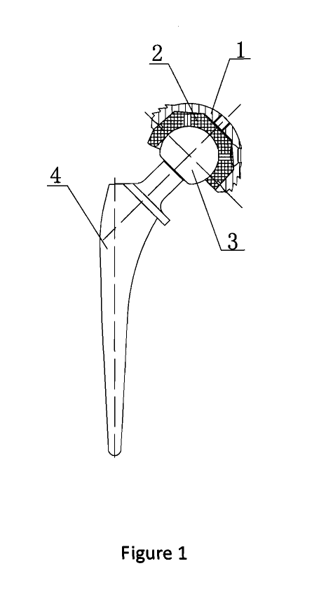

FIG. 2. Schematic Diagram for the Motion Limit of One Side of Traditional Total Hip Joint Prosthesis

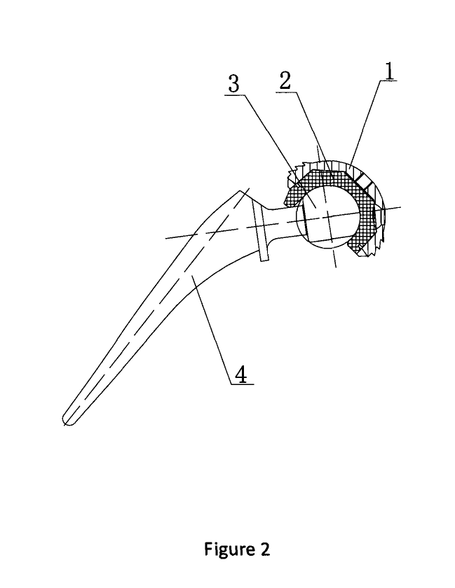

FIG. 3. Schematic Diagram for the Motion Limit of the Other Side of Traditional Total Hip Joint Prosthesis

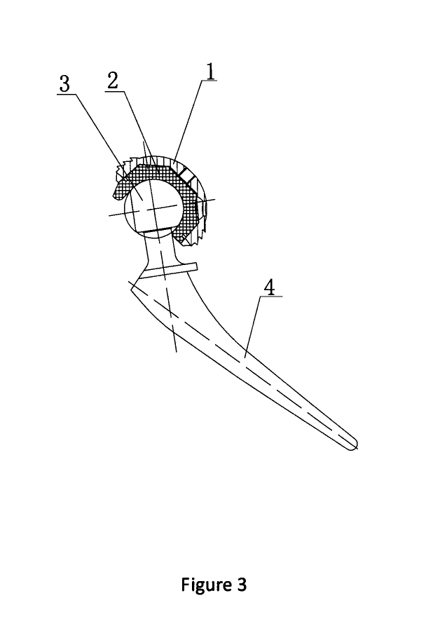

FIG. 4. Front View of This Invention

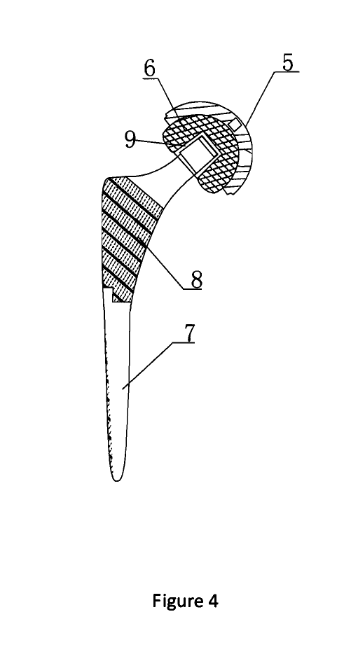

FIG. 5. Schematic Diagram for the Motion Limit of This Invention

The figure presents metal acetabular cup 1, femoral head 2, lining 3, femoral stem 4, acetabular cup prosthesis 5, non-metal femoral head prosthesis 6, femoral stem prosthesis 7, rough surface 8 and tapered inner core 9.

### SPECIFIC IMPLEMENTATION MODE

To help the technicians in this technical field better understand the technical scheme, further explanations on the invention are made according to the following figures and best implementation examples.

As revealed by the figures, this invention consists of acetabular cup prosthesis 5, non-metal femoral head prosthesis 6 and femoral stem prosthesis 7. The acetabular cup prosthesis has an inner spherical surface and the non-metal femoral head prosthesis which moves within the acetabular cup prosthesis is equipped with a tapered inner core. A tapered locked connection will take shape when the femoral stem prosthesis is inserted into the tapered inner core of the non-metal femoral head prosthesis.

The tapered inner core is preferably made of metal or ceramic.

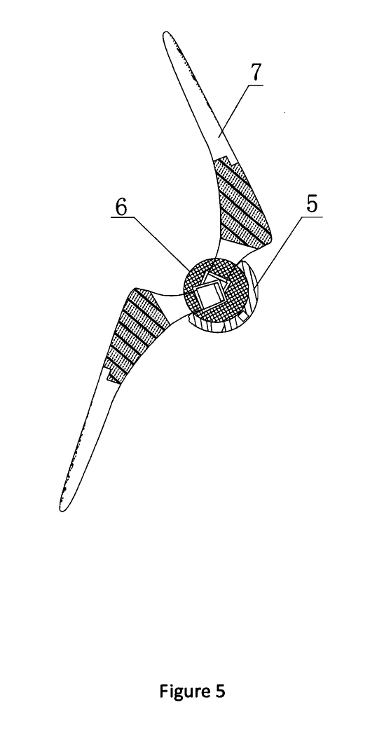

FIG. 5 shows the motion limit of two sides of the non-metal ball-headed hip joint prosthesis. Due to the combination of lining with femoral head, it is unnecessary to make one side of lining protrude from metal acetabular cup so that the limitations on the motion range of joint prosthesis will be removed.

In this invention, the combination of original lining with femoral head has reduced the connections between different parts to remarkably reduce the probability of abrasion and make up for the deficiency that traditional total joint prosthesis has a small motion range.

The non-metal femoral head prosthesis is made of highly abrasion-resistant non-metal materials, and the preferable choice is highly crosslinked polyethylene or polyether ether ketone (PEEK).

A rough surface may be designed for femoral stem prosthesis in order to ensure the stability after the implantation of joint prosthesis.

To address the problem that the untimely ingrowth of femoral stem prosthesis at the inner side after the implantation, the rough surface at the outside of femoral stem prosthesis may undergo the cutting process at the right angle so that force transmission will begin from the inner side to effectively promote the ingrowth of bone.

The above is the description on the preferable implementation mode of this invention. It is worth noticing that ordinary technicians in this technical domain may also make improvements which should be also deemed to fall under the scope of this invention on the premise of following the principle of this invention.

1. The non-metal ball-headed hip joint prosthesis includes acetabular cup, non-metal femoral head and femoral stem prostheses. The acetabular cup prosthesis has an inner spherical surface, which matches the external spherical surface of non-metal femoral head prosthesis. The non-metal femoral head prosthesis which moves within the acetabular cup prosthesis is equipped with a tapered inner core. A tapered locked connection will take shape when the femoral stem prosthesis is inserted into the tapered inner core of the non-metal femoral head prosthesis.

2. As described by claim 1, in the non-metal ball-headed hip joint prosthesis, the femoral stem prosthesis has a rough surface.

3. As described by claim 1, in any non-metal ball-headed hip joint prosthesis, the rough surface at the outside of the femoral stem prosthesis undergoes the cutting process at its right angle.

4. As described by claim 1, in any non-metal ball-headed hip joint prosthesis, the non-metal femoral head prosthesis is made of highly crosslinked polyethylene.

5. As described by claim 1, in any non-metal ball-headed hip joint prosthesis, the non-metal femoral head prosthesis is made of polyether ether ketone (PEEK).

6. As described by claim 1, in any non-metal ball-headed hip joint prosthesis, the tapered inner core is made of metal.

7. As described by claim 1, in any non-metal ball-headed hip joint prosthesis, the tapered inner core is made of ceramic.
